(19) United States
(12) Patent Application Publication
(10) Pub. No.: US 2024/0238284 A1
(43) Pub. Date: Jul. 18, 2024

(54) COMPOUNDS AND COMPOSITIONS FOR THE TREATMENT OF MPNST

(71) Applicants: Novartis AG, Basel (CH); The John Hopkins University, Baltimore, MD (US)

(72) Inventors: Huaixiang HAO, Lexington, MA (US); Susan Elizabeth MOODY, Cambridge, MA (US); Christine A. PRATILAS, Woodbine, MD (US); Jiawan WANG, Baltimore, MD (US)

(21) Appl. No.: 18/558,756

(22) PCT Filed: Apr. 28, 2022
(86) PCT No.: PCT/IB2022/053970

§ 371 Date: Nov. 3, 2023

Related U.S. Application Data
(60) Provisional application No. 63/184,422, filed on May 5, 2021.

(51) Int. Cl. A61K 31/497; A61K 31/517; A61P 35/00

(52) U.S. Cl. CPC A61K 31/497; A61K 31/517; A61P 35/00

(57) ABSTRACT

The present invention relates to a pharmaceutical combination comprising a SHP2 inhibitor and a CDK4/6 inhibitor; pharmaceutical compositions comprising the same; and methods of using such combinations and compositions in the treatment or prevention of conditions in which a SHP2 inhibitor combined with CDK4/6 inhibition is beneficial in, for example, the treatment of NF-1 associated MPNST.

### FIELD OF THE INVENTION

The present invention relates to a SHP2 inhibitor, a pharmaceutical combination comprising a SHP2 inhibitor and a CDK4/6 inhibitor and a pharmaceutical combination comprising a MEK inhibitor and a CDK4/6 inhibitor; pharmaceutical compositions comprising the same; and methods of using such compounds, combinations and compositions in the treatment of conditions in which SHP2 inhibition or CDK4/6 inhibition combined with SHP2 inhibition or MEK inhibition is beneficial, for example, in the treatment of malignant peripheral nerve sheath tumors (MPNST).

### BACKGROUND OF THE INVENTION

Malignant peripheral nerve sheath tumors (MPNSTs) are rare, aggresive soft-tissue sarcomas with high unmet clinical need, especially in the metastatic or unresectable setting. No standard therapy exists for this indication, although soft tissue sarcoma chemotherapy regimens can provide limited benefit. MPNSTs can occur either sporadically (˜45%), in association with neurofibromatosis type 1 (˜45%), or in association with prior radiotherapy (˜10%). Neurofibromatosis type 1 (NF1) is a common neurogenetic syndrome characterized by neurocognitive effects, a predisposition to develop benign and malignant tumors, cutaneous and other physical findings, and, in 30-50% of patients, plexiform neurofibromas (pNF). pNF are precursor tumors to the malignant counterpart, malignant peripheral nerve sheath tumor (MPNST), and can themselves be a substantial cause of pain, disfigurement and dysfunction.

TNO155 is an orally bioavailable, allosteric inhibitor of Src homology-2 domain containing protein tyrosine phsophatase-2 (SHP2, encoded by the PTPN11 gene), which transduces signals from activated receptor tyrosine kinases (RTKs) to downstream pathways, including the extracellular signal-regulated kinase (ERK) pathway. SHP2 has also been implicated in immune checkpoint and cytokine receptor signaling. TNO155 has demonstrated efficacy in a wide range of RTK-dependent human cancer cell lines and in vivo tumor xenografts.

Cyclin D proteins are critical in cancer cell division and complex with the CDK4 and CDK6 protein kinases to promote G1 to S phase cell cycle progression by hyperphosphorylating and activating the retinoblastoma protein (Rb). Ribociclib inhibits CDK4/6 specific phosphorylation of Rb, thereby halting cell cycle progression in the G1 phase. Cyclin DI is an effector of signaling downstream of mutant EGFR and other RTKs, suggesting that the cyclin DI-CDK4/6 axis plays an important role in proliferation downstream of RTKs.

SHP2 inhibition, combinations of SHP2 and CDK4/6 inhibitors, or combinations of CDK4/6 and MEK inhibitors are active and produce durable responses in MPNST, representing a novel treatment strategy for patients with metastatic or unresectable MPNST.

### SUMMARY OF THE INVENTION

The present invention provides for a SHP2 inhibitor for the treatment of metastatic or unresectable MPNST.

In another embodiment, the present invention provides for a pharmaceutical composition comprising:
(a) a SHP2 inhibitor and (b) a CDK4/6 inhibitor, for the treatment of MPNST.

In another embodiment, the present invention provides for a pharmaceutical composition comprising:
(a) a CDK4/6 inhibitor and (b) a MEK inhibitor, for the treatment of MPNST.

A combination of either a SHP2i+CDK4/6i or CDK4/6i+MEKi, will also be referred to herein as a “combination of the invention”.

In another embodiment of the combinations of the invention, a SHP2i+CDK4/6i or a CDK4/6i+MEKi are in the same formulation.

In another embodiment of the combinations of the invention, a SHP2i+CDK4/6i or a CDK4/6i+MEKi are in separate formulations.

In another embodiment, a combination of the invention is for simultaneous or sequential (in any order) administration.

In another embodiment is a method for treating MPNST (sporadic MPNST, or NF1-associated MPNST or MPNST associated with radiotherapy) in a patient in need thereof comprising administering to the patient a therapeutically effective amount of a SHP2i or the combination of either a SHP2i+CDK4/6i or a CDK4/6i+MEKi.

In a further embodiment, a SHP2i or the combination of either a SHP2i+CDK4/6i or a CDK4/6i+MEKi provides for a use in the manufacture of a medicament for treating MPNST (sporadic MPNST, or NF1-associated MPNST or MPNST associated with radiotherapy) in a patient in need thereof.

In another embodiment is a pharmaceutical composition comprising the combinations of the invention.

In a further embodiment, the pharmaceutical composition further comprises one or more pharmaceutically acceptable excipients.

### BRIEF DESCRIPTION OF THE DRAWINGS

FIG. 1 shows the results for the combination of SHP2i (TNO155)+CDK4/6i (ribociclib) in PDX models JH-2-031, WU-225, WU-386, WU-545, JH-2-079 and JH-2-002.

FIG. 2 shows the results for the combination of CDK4/6i (ribociclib) and MEKi (trametinib) in PDX models JH-2-031, WU-225, WU-386, WU-545 and JH-2-079.

FIG. 3 shows results (as a heat map) for the combination of CDK4/6i (ribociclib)+SHP2i (TNO155) in ten native NF1-MPNST cell lines and two trametinib resistant cell lines (ST8814Res and NF90.8Res).

FIG. 4 shows results for the combination of CDK4/6i (ribociclib)+SHP2i (TNO155) in 11 NF1-MPNST cell lines.

### DEFINITIONS

The general terms used hereinbefore and hereinafter preferably have within the context of this disclosure the following meanings, unless otherwise indicated, where more general terms wherever used may, independently of each other, be replaced by more specific definitions or remain, thus defining more detailed embodiments of the invention:

The term “treating” or “treatment” as used herein comprises a treatment relieving, reducing or alleviating at least one symptom in a patient or effecting a delay of progression of a disease. For example, treatment can be the diminishment of one or several symptoms of a disorder or complete eradication of a disorder, such NF-1 associated MPNST. Within the meaning of the present disclosure, the term “treat” also denotes to arrest, delay the onset (i.e., the period prior to clinical manifestation of a disease) and/or reduce the risk of developing or worsening a disease.

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “combination therapy” or “in combination with” refers to the administration of two or more therapeutic agents to treat a condition or disorder described in the present disclosure (e.g., sporadic MPNST or NF1-associated MPNST or associated with radiotherapy). Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

The term “SHP2i” includes, but is not limited to, TNO155, JAB-3068, JAB-3312, RMC-4630 (or any SHP2 inhibitors contained in U.S. Pat. No. 10,590,090), RLY-1971, BBP-398 (IACS-15509), ERAS-601 and PF-07284892 (ARRY-558).

The terms “CDK4/6i” includes, but is not limited to, ribociclib, palbociclib and abemaciclib.

The term “MEKi” includes, but is not limited to, trametinib, cobimetinib, binimetinib, mirdametinib, and selumetinib.

The term “synergistic effect” as used herein refers to action of two therapeutic agents such as, for example, a compound TNO155 as a SHP2 inhibitor and ribociclib as a CDK4/6 inhibitor, producing an effect, for example, slowing the symptomatic progression of NF-associated MPNST, or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

A particular combination of the invention, for example, TNO155 and ribociclib, is also intended to represent unlabeled forms as well as isotopically labeled forms of the compounds. Isotopically labeled compounds have one or more atoms replaced by an atom having a selected atomic mass or mass number. Examples of isotopes that can be incorporated into TNO155 and ribociclib include isotopes of hydrogen, carbon, nitrogen, oxygen, and chlorine, for example, ²H, ³H, ¹¹C, ¹³C, ¹⁴C, ¹⁵N, ³⁵S, ³⁶Cl. The invention includes isotopically labeled TNO155 and ribociclib, for example into which radioactive isotopes, such as ³H and ¹⁴C, or non-radioactive isotopes, such as ²H and ¹³C, are present. Isotopically labelled TNO155 and ribociclib are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. Isotopically-labeled compounds of the invention can generally be prepared by conventional techniques known to those skilled in the art or by processes analogous to those described in the accompanying Examples using appropriate isotopically-labeled reagents.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of either TNO155 or ribociclib. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in TNO155 or ribociclib is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### DESCRIPTION OF SPECIFIC EMBODIMENTS

In one embodiment is a method of treating a malignant peripheral nerve sheath tumor comprising administering to a patient in need thereof a therapeutically effective amount of a SHP2 inhibitor.

In a further embodiment, the malignant peripheral nerve sheath tumor is metastatic, unresectable, sporadic, associated with neurofibromatosis type 1 or associated with radiotherapy.

In a further embodiment, the SHP2 inhibitor is selected from TNO155, SHP099, JAB-3068, JAB-3312, RMC-4630 (or any SHP2 inhibitors contained in U.S. Pat. No. 10,590,090), RLY-1971, BBP-398 (IACS-15509), ERAS-601 and PF-07284892 (ARRY-558).

In a further embodiment, the SHP2 inhibitor is (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof.

In a further embodiment, the pharmaceutical salt is succinate.

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine is administered orally at a dose of about 1.5 mg per day, or 3 mg per day, or 6 mg per day, or 10 mg per day, or 20 mg per day, or 30 mg per day, or 40 mg per day, or 50 mg per day, or 60 mg per day, or 70 mg per day, or 80 mg per day, or 90 mg per day, or 100 mg per day.

In a further embodiment, the dosing schedule is selected from continuous, 2 weeks on/1 week off or 3 weeks on/1 week off.

In another embodiment is a method of treating a malignant peripheral nerve sheath tumor comprising administering to a patient in need thereof a pharmaceutical composition comprising: (a) a SHP2 inhibitor; and (b) a CDK4/6 inhibitor.

In a further embodiment, the malignant peripheral nerve sheath tumor is sporadic, associated with neurofibromatosis type 1 or associated with radiotherapy.

In a further embodiment, the SHP2 inhibitor is selected from TNO155, SHP099, JAB-3068, JAB-3312, RMC-4630 (or any SHP2 inhibitors contained in U.S. Pat. No. 10,590,090), RLY-1971, BBP-398 (IACS-15509), ERAS-601 and PF-07284892 (ARRY-558).

In a further embodiment, the SHP2 inhibitor is (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof.

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine is administered orally at a dose of about 1.5 mg per day, or 3 mg per day, or 6 mg per day, or 10 mg per day, or 20 mg per day, or 30 mg per day, or 40 mg per day, or 50 mg per day, or 60 mg per day, or 70 mg per day, or 80 mg per day, or 90 mg per day, or 100 mg per day.

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine is administered orally once daily wherein the dosing schedule is selected from continuous, 2 weeks on/1 week off or 3 weeks on/1 week off.

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine is administered orally once daily wherein the schedule is 2 weeks on and 1 week off treatment.

In a further embodiment, the CDK4/6 inhibitor is selected from ribociclib, palbociclib and abemaciclib.

In a further embodiment, the CDK4/6 inhibitor is 7-cyclopentyl-N,N-dimethyl-2-((5-(piperazin-1-yl)pyridin-2-yl)amino)-7H-pyrrolo[2,3-d]pyrimidine-6-carboxamide, or pharmaceutically acceptable salt thereof.

In a further embodiment, 7-cyclopentyl-N,N-dimethyl-2-((5-(piperazin-1-yl)pyridin-2-yl)amino)-7H-pyrrolo[2,3-d]pyrimidine-6-carboxamide is administered orally at a dose of about 100 mg per day, or 200 mg per day, or 300 mg per day, or 400 mg per day, or 500 mg per day, or 600 mg per day.

In a further embodiment, 7-cyclopentyl-N,N-dimethyl-2-((5-(piperazin-1-yl)pyridin-2-yl)amino)-7H-pyrrolo[2,3-d]pyrimidine-6-carboxamide is administered orally at 600 mg for 21 days followed by 7 days off treatment.

In another embodiment is a method of treating a malignant peripheral nerve sheath tumor comprising administering to a patient in need thereof a pharmaceutical composition comprising: (a) a MEK inhibitor; and (b) a CDK4/6 inhibitor.

In a further embodiment, the malignant peripheral nerve sheath tumor is sporadic, associated with neurofibromatosis type 1 or associated with radiotherapy.

In a further embodiment, the MEKi is selected from trametinib, cobimetinib, binimetinib, mirdametinib, and selumetinib.

In a further embodiment, the MEK inhibitor is N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide, or pharmaceutically acceptable salt thereof.

In a further embodiment, N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide is administered dimethyl sulfoxide per day is administered orally at a dose of about 0.5, 1, 1.5 and 2 mg daily.

In a further embodiment, the CDK4/6 inhibitor is 7-cyclopentyl-N,N-dimethyl-2-((5-(piperazin-1-yl)pyridin-2-yl)amino)-7H-pyrrolo[2,3-d]pyrimidine-6-carboxamide, or pharmaceutically acceptable salt thereof.

In a further embodiment, 7-cyclopentyl-N,N-dimethyl-2-((5-(piperazin-1-yl)pyridin-2-yl)amino)-7H-pyrrolo[2,3-d]pyrimidine-6-carboxamide is administered orally at a dose of about 100 mg per day, or 200 mg per day, or 300 mg per day, or 400 mg per day, or 500 mg per day, or 600 mg per day.

In a further embodiment, 7-cyclopentyl-N,N-dimethyl-2-((5-(piperazin-1-yl)pyridin-2-yl)amino)-7H-pyrrolo[2,3-d]pyrimidine-6-carboxamide is administered orally at 600 mg for 21 days followed by 7 days off treatment.

In another embodiment, the present invention provides for a SHP2 inhibitor selected from: (3S,4S)-8-(6-amino-5-((2-amino-3-chloropyridin-4-yl)thio)pyrazin-2-yl)-3-methyl-2-oxa-8-azaspiro[4.5]decan-4-amine (TNO155), or a pharmaceutically acceptable salt thereof, having the structure:

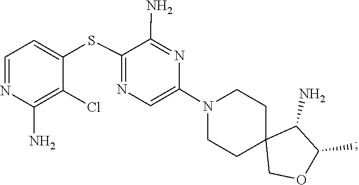 [SMILES: C[C@@H]1OCC2(CCN(c3cnc(Sc4ccnc(N)c4Cl)c(N)n3)CC2)[C@@H]1N]

for the treatment of MPNST (sporadic MPNST or MPNST associated with NF1 or associated with radiotherapy).

In another embodiment, the present invention provides for a pharmaceutical composition comprising:
(a) a SHP2 inhibitor selected from: (3S,4S)-8-(6-amino-5-((2-amino-3-chloropyridin-4-yl)thio)pyrazin-2-yl)-3-methyl-2-oxa-8-azaspiro[4.5]decan-4-amine (TNO155), or a pharmaceutically acceptable salt thereof, having the structure:

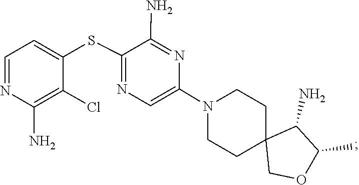 [SMILES: C[C@@H]1OCC2(CCN(c3cnc(Sc4ccnc(N)c4Cl)c(N)n3)CC2)[C@@H]1N]

and
(b) 7-cyclopentyl-N,N-dimethyl-2-((5-(piperazin-1-yl)pyridin-2-yl)amino)-7H-pyrrolo[2,3-d]pyrimidine-6-carboxamide (ribociclib), or a pharmaceutically acceptable salt thereof, having the structure:

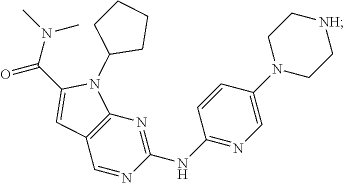 [SMILES: CN(C)C(=O)c1cc2cnc(Nc3ccc(N4CCNCC4)cn3)nc2n1C1CCCC1]

for the treatment of MPNST (sporadic MPNST or MPNST associated with NF1 or associated with radiotherapy).

In another embodiment, the present invention provides for a pharmaceutical composition comprising:
(a) 7-cyclopentyl-N,N-dimethyl-2-((5-(piperazin-1-yl)pyridin-2-yl)amino)-7H-pyrrolo[2,3-d]pyrimidine-6-carboxamide (ribociclib), or a pharmaceutically acceptable salt thereof, having the structure:

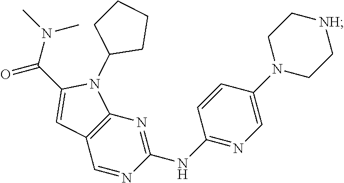 [SMILES: CN(C)C(=O)c1cc2cnc(Nc3ccc(N4CCNCC4)cn3)nc2n1C1CCCC1]

and
(b) N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib), or a pharmaceutically acceptable salt or solvate thereof, having the structure:

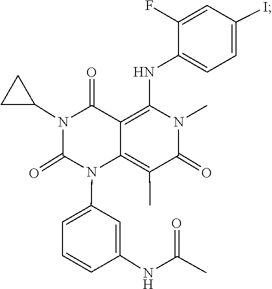 [SMILES: CC(=O)Nc1cccc(-n2c(=O)n(C3CC3)c(=O)c3c(Nc4ccc(I)cc4F)n(C)c(=O)c(C)c32)c1]

for the treatment of MPNST (sporadic MPNST or MPNST associated with NF1 or associated with radiotherapy).

In one embodiment is a method of treating MPNST (sporadic MPNST or MPNST associated with NF1 or associated with radiotherapy) comprising administering to a patient in need thereof a pharmaceutical composition comprising (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof.

In another embodiment is a method of treating MPNST (sporadic MPNST or MPNST associated with NF1 or associated with radiotherapy) comprising administering to a patient in need thereof a pharmaceutical composition comprising (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, in combination with a second therapeutic agent.

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, and the second therapeutic agent are administered simultaneously, separately or over a period of time.

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, administered to the patient in need thereof, is effective to treat MPNST (sporadic MPNST or MPNST associated with NF1 or associated with radiotherapy).

In a further embodiment, the method comprises a second therapeutic agent.

In a further embodiment, the amount of (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, and the second therapeutic agent, administered to the subject in need thereof, is effective to treat MPSNT (sporadic MPNST or MPNST associated with NF1 or associated with radiotherapy).

In a further embodiment, the second therapeutic agent is a CDK4/6 inhibitor.

In a further embodiment, the CDK4/6 inhibitor is 7-cyclopentyl-N,N-dimethyl-2-((5-(piperazin-1-yl)pyridin-2-yl)amino)-7H-pyrrolo[2,3-d]pyrimidine-6-carboxamide, or a pharmaceutically acceptable salt thereof.

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine is administered orally at a dose of about 1.5 mg per day, or 3 mg per day, or 6 mg per day, or 10 mg per day, or 20 mg per day, or 30 mg per day, or 40 mg per day, or 50 mg per day, or 60 mg per day, or 70 mg per day, or 80 mg per day, or 90 mg per day, or 100 mg per day

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine is administered orally at a dose per day of 20 mg on a 21 day cycle of 2 weeks on drug followed by 1 week off drug.

In a further embodiment, 7-cyclopentyl-N,N-dimethyl-2-((5-(piperazin-1-yl)pyridin-2-yl)amino)-7H-pyrrolo[2,3-d]pyrimidine-6-carboxamide is administered orally at a dose of about 100 mg per day, or 200 mg per day, or 300 mg per day, or 400 mg per day, or 500 mg per day, or 600 mg per day.

In a further embodiment, 7-cyclopentyl-N,N-dimethyl-2-((5-(piperazin-1-yl)pyridin-2-yl)amino)-7H-pyrrolo[2,3-d]pyrimidine-6-carboxamide is administered orally at 200 mg for 21 days.

In a further embodiment, 7-cyclopentyl-N,N-dimethyl-2-((5-(piperazin-1-yl)pyridin-2-yl)amino)-7H-pyrrolo[2,3-d]pyrimidine-6-carboxamide is administered orally at 300 mg for 21 days followed by 7 days off treatment.

In a further embodiment, 7-cyclopentyl-N,N-dimethyl-2-((5-(piperazin-1-yl)pyridin-2-yl)amino)-7H-pyrrolo[2,3-d]pyrimidine-6-carboxamide is administered orally at 600 mg for 21 days followed by 7 days off treatment.

In another embodiment is a method of treating MPNST (sporadic MPNST or MPNST associated with NF1 or associated with radiotherapy) comprising administering to a patient in need thereof a pharmaceutical composition comprising (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, in combination with ribociclic to overcome MEKi (trametinib) resistance.

In another embodiment is a method of treating MPNST (sporadic MPNST or MPNST associated with NF1 or associated with radiotherapy) comprising administering to a patient in need thereof a pharmaceutical composition comprising (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, in combination with ribociclic for MPNST patients who have received MEK inhibitor treatment for their prior benign tumor neurofibromas and have developed acquired resistance.

In another embodiment is a method of treating MPNST (sporadic MPNST or MPNST associated with NF1 or associated with radiotherapy) comprising administering to a patient in need thereof a pharmaceutical composition comprising (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, in combination with ribociclic for MPNST patients who exhibit intrinsic resistance to MEK inhibitor.

### Pharmacology and Utility

Neurofibromatosis type 1 (NF1) is one of the most common inherited tumor predisposition syndromes, affecting 1:2500-3000 individuals worldwide. As such, affected individuals begin life with one inactivated copy (germline mutation) and one functional copy of the NF1 gene in every cell in their body. The presence of the germline mutation increases the risk of tumor formation, which requires only somatic loss of the remaining functional NF1 gene.

NF1 is characterized by neurocognitive effects, a predisposition to develop benign and malignant tumors, cutaneous and other physical findings, and, in 30-50% of patients, plexiform neurofibromas (pNF). pNF are precursor tumors to the malignant counterpart, malignant peripheral nerve sheath tumor (MPNST), and can themselves be a substantial cause of pain, disfigurement and dysfunction. The overall lifetime risk of transformation from pNF to MPNST approaches 10%, and NF1 patients develop MPNST at a significantly younger age than those with spontaneous MPNST.

MPNSTs are rare, aggresive soft-tissue sarcomas with high unmet clinical need, especially in the metastatic or unresectable setting. No standard therapy exists for this indication, although soft tissue sarcoma chemotherapy regimens can provide limited benefit. The challenges associated with treating patients with MPNST include their relative insensitivity to conventional systemic chemotherapy and radiotherapy, and their propensity to metastasize. The only known definitive therapy for MPNST is surgical resection with wide negative margins, which is often not feasible due to location or size, the associated morbidity of the surgery, or the presence of distant metastases. Despite many clinical trials of chemotherapy and targeted agents, there has been little advancement in overall patient survival. A retrospective pooled analysis of 12 studies in which various such regimens were used as initial therapy (i.e., first-line) demonstrated a response rate of 21%, median progression-free survival of 17 weeks, and median overall survival of 48 weeks. Given the limited benefit of chemotherapy, a number of molecularly targeted therapies have been explored in patients with MPNST, with dismal results. Recent clinical trials of such agents have consistently achieved objective response rates of 0% and, where reported, median overall survival was approximately 4-5 months.

MPNSTs are commonly characterized by loss of the tumor suppressor NF1, and their incidence is enriched in patients with the autosomal dominant NF1 germline loss cancer predisposition syndrome, Neurofibromatosis Type 1 (NF1). Patients with NF1 syndrome are estimated to have an 8-13% lifetime incidence of MPNST and an annual incidence of 1.6 per 1000 individuals, while the incidence of sporadic MPNST in the general population in the United States is 1.46 per million person-years.

Among all MPNSTs, approximately 22-50% arise in patients with germline NF1 loss, while the remainder arise sporadically. Prior radiation therapy is a risk factor for MPNST, with approximately 10% of MPNSTs arising in this setting. All NF1-associated MPNSTs and the majority of those arising sporadically or in the setting of prior radiotherapy are characterized by loss of NF1, and the second most commonly detected genetic alteration in each of these settings is loss of the tumor suppressor. In patients with germline NF1 loss, loss of CDKN2A is considered to be an early step in malignant progression, occurring at the transition of benign plexiform neurofibromas to atypical neurofibromas, which are precursors to MPNSTs.

Neurofibromin, the gene product of NF1, is a RAS GTPase-activating protein (RAS-GAP) involved in the hydrolysis of active RAS-GTP to inactive RAS-GDP. It is genetically altered in nearly 90% of MPNST. Accordingly, aberrant RAS activation underlies the pathogenesis of NF1-mutant cancers. It is not known, however, whether a single RAS family member is the predominant RAS which is activated in NF1-deficient MPNST, nor is the extent of functional redundancy of the classic RAS family members HRAS, NRAS and KRAS well-understood in this tumor type. Among the well-characterized RAS effector pathways are RAF/MEK/ERK, PI3K/AKT and Ral-GDS signaling. Of these, ERK signaling is a critical downstream effector and thus the concept of pharmacological MEK inhibition has been applied to models of MPNST. The MEK inhibitor (MEKi) selumetinib results in partial responses in 71% of children with NF1-related pNF (NCT01362803). The preclinical responses of MPNST to single agent MEKi, however, have been partial. This suggests a need for improved understanding of the role of ERK and other RAS effector pathways. Additional signaling pathways have been implicated in MPNST tumorigenesis, including mTOR signaling, and pharmacological inhibition of these pathways has been proposed. Further, inactivation of the polycomb repressive complex-2 (PRC2) via loss of function (LOF) of SUZ12 or EED recurrently and specifically occurs in MPNST, but not in its benign counterpart pNF, and has been implicated in amplification of RAS-driven transcription. A complex cooperation between inactivation of tumor suppressors and activation of oncogenic pathways likely occurs in NF1-driven tumorigenesis and inhibiting more than one RAS effector pathway may be necessary for complete anti-tumor effects.

NF1 gene inactivation and loss of NF1 protein (neurofibromin) expression characterize the majority of NF1-MPNSTs. While NF1 loss is necessary for MPNST development, it is not sufficient for malignant transformation. About 50% of MPNSTs are sporadic (i.e., arise in patients without germline NF1 loss and thus without the NFL syndrome). Most of the sporadic MPNSTs have somatic NF1 loss in the tumor.

Alterations in the TP53. CDKN2A. and EED SUZ12 genes have been reported as cooperating secondary genetic alterations that promote MPNST development. Molecular targeting of each of these LOF alterations, however, represents a unique challenge. Further, transcriptional analysis studies have revealed upregulated expression of cell cycle-promoting genes including RABL6A, a negative regulator of RB1. Loss of CDKN2A (the gene encoding p16 INK4a), inactivation of RB, and hyper-activation of cyclin dependent kinases (CDK) suggest that small-molecule CDK4/6 inhibitors (CDK4/6i) may be a therapeutic strategy. Monotherapy with CDK4/6i, however, exhibits limited efficacy due to bypass mechanisms such as CDK2 hyper-activation and E2F amplification. Other studies suggest upregulation of cell cycle regulators aurora kinase A (AURKA) and polo-like kinase (PLK1), but single agent treatment with aurora kinase or PLK1 inhibitors has a narrow therapeutic index, modest in vivo anti-tumor activity, and no objective responses observed in human trials. Furthermore, combined CDK4/6i and MEKi has demonstrated synergistic effects in preclinical models of melanoma, neuroblastoma, and pancreatic and KRAS-mutant colorectal cancers. Given the dependency of D-cyclins on RAS signaling and recurrent loss of CDKN2A in MPNST, the cytostatic effects of CDK4/6i may be potentiated to induce apoptosis together with drugs targeting upstream RTK/regulators of RAS (SHP2) or downstream RAS signaling such as ERK pathway (MEKi) in MPNST.

MEK inhibitors alone are ineffective in MPNST, which prompts the exploration of combinatorial therapeutics using MEKi and agents targeting the adaptively changed signaling elements that emerge upon short-term MEK inhibition. “Adaptive resistance” to MEK and other small-molecule inhibitors involves dynamic changes in signaling networks and non-genomic bypass mechanisms that occur frequently via transcriptional induction of genes for receptor tyrosine kinases (RTK) or their ligands, leading to a transient and partial response. Inability to predict which RTK will become critically upregulated as a signaling adaptation to MEKi represents a challenge in designing combination therapy of MEKi+RTKi.

In the setting of NF1 loss, the RAS-MAPK pathway, a well-validated oncogenic driver, is hyperactivated due to this impairment of RAS inactivation. SHP2 is a cytoplasmic phosphatase that is involved in RAS GTP loading, accelerating the transition of RAS from the inactive GDP-bound state to the active GTP-bound state. Therefore, inhibition of SHP2 is anticipated to counter the RAS-activating effect of NF1 loss.

A strategy co-targeting a signaling node that represents a point of convergence from upstream RTK signaling, together with inhibition of RAS effector pathways therefore becomes necessary and PTPN11/SHP2 phosphatase represents such a promising target. SHP2 is a central node in adaptive resistance driven by RTK reactivation and MEKi in multiple cancer models. SHP2 phosphatase facilitates RAS-GEF-mediated RAS-GTP loading, accelerating the transition of RAS from the inactive GDP-bound state to the active GTP-bound state, and recruitment of RAS to the cell membrane, where RTK activation occurs, and therefore is required for RAS/ERK pathway activation by most RTK. SHP2 inhibition counteracts the RAS-activating effects of NF1 loss. NF1 is involved in de-activating RAS, while SHP2 is involved in activating RAS.

Thus, SHP2 inhibition (SHP2i) and combination SHP2i can be a strategy to overcome signaling adaptation to, for example, MEKi in tumors with hyperactive RAS due to loss of NF1. There is a need to design rational combination therapies that inhibit inhibitor-induced pathway reactivation to identify optimal therapeutic strategies to effectively target NF1-associated MPNST.

TNO155 is a first-in-class allosteric inhibitor of wild-type SHP2. SHP2 is a ubiquitously expressed non-receptor protein tyrosine phosphatase (PTP) composed of two N-terminal SH2 domains, a classic PTP domain, and a C-terminal tail. The phosphatase activity is auto-inhibited by the two SHP2 domains that bind to the PTP domain (closed conformation). Upon activation of receptor tyrosine kinases (RTKs), SHP2 is recruited to the plasma membrane where it associates with activated RTKs and a number of adaptor proteins to relay signaling by activating the RAS/ERK pathway. TNO155 binds the inactive, or “closed” conformation of SHP2, thereby preventing its opening into the active conformation. This prevents the transduction of signaling from activated RTKs to the downstream RAS/ERK pathway.

TNO155 has demonstrated efficacy in a wide range of RTK-dependent human cancer cell lines and in vivo xenografts. SHP2 inhibition can be measured by assessing biomarkers within the ERK signaling pathway, such as decreased levels of phosphorylated ERK1/2 (pERK) and downregulation of dual specificity phosphatase 6 (DUSP6) mRNA transcript. In the KYSE-520 (esophageal squamous cell carcinoma) and DETROIT-562 (pharyngeal squamous cell carcinoma) cancer cell lines, the in vitro pERK IC50's were 8 nM (3.4 ng/mL) and 35 nM (14.8 ng/mL) and the antiproliferation IC50's were 100 nM (42.2 ng/mL) and 470 nM (198.3 ng/mL), respectively. The antiproliferative effect of TNO155 was revealed to be most effective in cancer cell lines that are dependent on RTK signaling. In vivo, SHP2 inhibition by orally-administered TNO155 (20 mg/kg) achieved approximately 95% decrease in DUSP6 mRNA transcript in an EGFR-dependent DETROIT-562 cancer cell line and 47% regression when dosed on a twice-daily schedule. Dose fractionation studies, coupled with modulation of the tumor DUSP6 biomarker show that maximal efficacy is achieved when 50% PD inhibition is attained for at least 80% of the dosing interval. Given the extensive cross-talk between the ERK pathway and the CDK4/6 complex in cancer cells, the combinations of TNO155 with the selective CDK4/6 inhibitor, ribociclib, was explored.

Ribociclib (LEE011, Kisqali®) is an orally bioavailable, highly selective small molecule inhibitor of cyclin-dependent kinases 4 and 6 (CDK4/6). Ribociclib has been approved by a number of Health Authorities, including the United States Food and Drug Administration (U.S. FDA) and the European Commission, as an initial endocrine-based therapy for the treatment of postmenopausal women with hormone receptor (HR)-positive, human epidermal growth factor receptor 2 (HER2)-negative advanced or metastatic breast cancer in combination with an aromatase inhibitor (AI) based on a randomized, double-blind, placebo-controlled, international clinical trial (MONALEESA-2 [CLEE011A2301]). On 18 Jul. 2018, the U.S. FDA expanded the indication for ribociclib in combination with an AI to include pre/perimenopausal women with HR-positive, HER2-negative advanced or metastatic breast cancer, as initial endocrine-based therapy. The expanded indication also includes ribociclib in combination with fulvestrant for postmenopausal women with HR-positive, HER2-negative advanced or metastatic breast cancer, as initial endocrine-based therapy or following disease progression on endocrine therapy (MONALEESA-7 [CLEE011E2301] and MONALEESA-3 [CLEE011F2301], respectively). Additional marketing authorizations in HR-positive, HER2-negative advanced or metastatic breast cancer are under review by health authorities worldwide. Additional phase III clinical trials for the treatment of HR-positive breast cancer patients, as well as several other phase I or II clinical studies are being conducted.

Ribociclib inhibits the CDK4/Cyclin DI and CDK6/Cyclin D3 enzyme complexes with IC50 values of 0.01 and 0.039 μM in biochemical assays, respectively, while showing a high degree of selectivity for CDK4/6 versus other cyclin-dependent kinases. In more than 40 Rb-positive cell lines derived from diverse cancer types, ribociclib inhibited Retinoblastoma protein (Rb) phosphorylation and interfered with G1 to S phase cell cycle progression. In contrast, in lineage-matched Rb-negative cell lines no effect of ribociclib on cell cycle progression was observed.

Ribociclib has demonstrated in vivo anti-tumor activity in subsets of tumor xenograft models including but not limited to breast, melanoma, neuroblastoma, malignant rhabdoid, lung, pancreas and hematological malignancies. In addition, ribociclib has shown anti-tumor activity when combined with targeted agents which inhibit signaling pathways known to regulate Cyclin D levels, including inhibitors of the RAF, mitogen-activated protein kinase kinase (MEK), phosphoinositide 3-kinase (PI3K) and mammalian target of rapamycin (mTOR) pathways.

TNO155 is currently being investigated in combination with ribociclib in study CTNO155B12101.

Current treatment options for NF1-MPNST are limited, with surgery for localized disease and radiotherapy to reduce the risk of local recurrence. The only prospective study using cytotoxic chemotherapy demonstrated a response rate of ˜17% (5/29 subjects) following doxorubicin and ifosfamide treatment in patients with NF1, suggesting that MPNST are minimally responsive to chemotherapy. Similar responses have been reported using various chemotherapy agents for metastatic disease. In addition, numerous molecularly-targeted therapies, which were highly effective in murine Nf1 MPNST preclinical studies, have proven ineffective when translated to human clinical trials. It is believed that the poor translation of preclinical findings to the bedside reflects two major barriers: (1) current preclinical models do not reflect the spectrum of genetic heterogeneity observed in human MPNST and (2) treatments may exhibit variable efficacy based on molecular subtypes, which are currently not captured using a single genetic model. By generating patient-derived models, the spectrum of molecular subtypes of MPNST can be better characterized to determine how various MPNST will respond to therapies.

To address this critical problem, a series of patient-derived MPNST xenografts have been generated that more broadly reflect the genetic heterogeneity seen in the human condition. The preference to generate PDX lines instead of traditional cell lines has been guided by two scientific principles. First, PDX lines have been shown at early passages to mirror the parental tumors. Second, the PDX lines are thought to be less susceptible to genetic drift than traditional cell lines, partially because they are not grown on plastic and forced to adapt to growth outside of a host.

Example 1, below, makes use of generated NF1-MPNST patient-derived xenograft (PDX) lines propagated in immunocompromised NRG or NSG mice. They harbor the spectrum of genomic alterations that are seen in patients with NF1, including germline and somatic NF1 mutations, as well as loss of CDKN2A. TP53 mutations. EED SUZ12 mutations, and numerous copy number changes. The preclinical data presented in example 1, below, provides evidence that the combination of the SHP2 inhibitor, TNO155, and the CDK4/6 inhibitor, ribociclib, exert a combination benefit in NF-1 associated MPNST.

### Pharmaceutical Compositions

In another aspect, the present invention provides pharmaceutically acceptable compositions which comprise a therapeutically-effective amount TNO155 and ribociclib, formulated together with one or more pharmaceutically acceptable carriers (additives) and/or diluents. As described in detail below, the pharmaceutical compositions of the present invention may be specially formulated for administration in solid or liquid form, including those adapted for oral administration, for example, drenches (aqueous or non-aqueous solutions or suspensions), tablets, e.g., those targeted for buccal, sublingual, and systemic absorption, boluses, powders, granules, pastes for application to the tongue.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxy benzoic, fumaric, toluenesulfonic, methanesulfonic, ethane disulfonic, oxalic, isothionic, and the like. The pharmaceutically acceptable salt of TNO155, for example, is succinate.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution, suspension or solid dispersion in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions.

When the compounds of the present invention are administered as pharmaceuticals to patients, they can be given per se or as a pharmaceutical composition containing, for example, 0.1 to 99% (more preferably, 10 to 30%) of active ingredient in combination with a pharmaceutically acceptable carrier.

The compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician having ordinary skill in the art can readily determine and prescribe the effective amount of the pharmaceutical composition required. For example, the physician could start doses of the compounds of the invention employed in the pharmaceutical composition at levels lower than that required in order to achieve the desired therapeutic effect and gradually increase the dosage until the desired effect is achieved.

In general, a suitable daily dose of the combination of the invention will be that amount of each compound which is the lowest dose effective to produce a therapeutic effect. Such an effective dose will generally depend upon the factors described above.

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents.

### EXAMPLES

### TNO155, Ribociclib and Trametinib

(3S,4S)-8-(6-amino-5-((2-amino-3-chloropyridin-4-yl)thio)pyrazin-2-yl)-3-methyl-2-oxa-8-azaspiro[4.5]decan-4-amine (TNO155) is synthesized according to example 69 of WO2015/107495. 7-cyclopentyl-N,N-dimethyl-2-((5-(piperazin-1-yl)pyridin-2-yl)amino)-7H-pyrrolo[2,3-d]pyrimidine-6-carboxamide (ribociclib) is synthesized according to example 74 of WO2010/020675. N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide (trametinib) is synthesized according to example 4-1 of WO2005/121142. WO2015/107495, WO2010/020675 and WO2005/121142/are herein incorporated by reference in their entirety.

SHP2, MEK and CDK4/6 are critical nodes in RAS effector signaling in MPNST, and combinations of inhibitors of these molecules can have synergistic anti-tumor activity. The in vivo anti-tumor effects and toxicity of TNO155 single agent and combination therapies (TNO155+Ribociclib), (Ribociclib+Trametinib), in PDX models were tested (Example 1).

### Example 1

### TNO155 Single Agent and Combination Benefits of TNO155+Ribociclib or Ribociclib and Trametinib in NF1-MPNST Patient-Derived Xenograft (PDX) Models

The PDX models of Example 1 were developed and characterized according to Dehrner, et al., JCI Insight 2021: 6(6):e146351 and Pollard, K., et al., Sci Data, 2020; 7:184). NRG (NOD-Rag1^{null} IL 2rg^{null}, NOD rag gamma) mice are used for all experiments.

SHP2i (TNO155 (A) 7.5 mg/kg) and CDK4/6i (ribociclib (B), 75 mg/kg, once daily (5 days per week), at half-human equivalent RP2D) and MEKi (trametinib (C) 0.075 mg/kg or 0.15 mg/kg once daily) and were given to NRG mice by oral gavage. Tumor response, survival and toxicity data were collected and analyzed. To form tumors, 1-2 million NF1-MPNST PDX-derived cells (JH-2-031, JH-2-079, JH-2-002, WU-225, WU-386, or WU-545)) in 50% Matrigel (BD Biosciences) were injected subcutaneously in 4- to 6-week-old female NRG mice. The tumors were measured and mice weighed twice weekly once tumors began to form. Drugs were given beginning when tumors reached 150-200 mm³. Mice were treated with vehicle, single agent (A or B or C) or combination (A+B or B+C) for up to 42 days. Each group consisted of three to five mice. Tumor volume was calculated using the formula: V=L×W2 (π/6), where L=longest diameter and W=width.

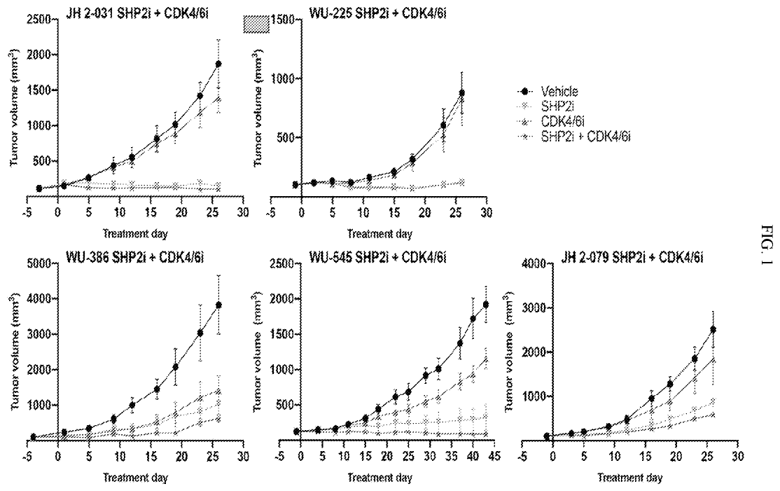

FIG. 1 shows the results for the combination of SHP2i (TNO155)+CDK4/6i (ribociclib) in PDX models JH-2-031, WU-225, WU-386, WU-545, JH-2-079 and JH-2-002.

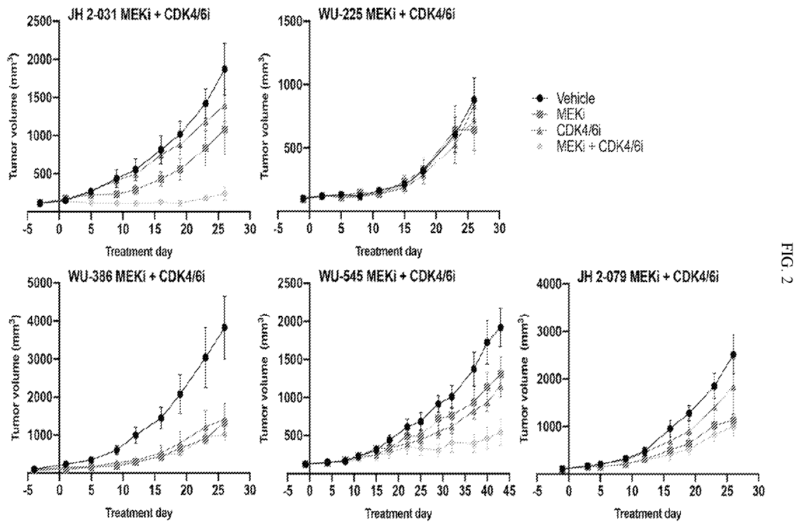

FIG. 2 shows the results for the combination of CDK4/6i (ribociclib) and MEKi (trametinib) in PDX models JH-2-031, WU-225, WU-386, WU-545 and JH-2-079.

Combination benefit was evident in the five in vivo PDX tested. Although some PDX models demonstrated similar responses to SHP2i alone or SHP2i+CDK4/6i during the initial 4 weeks on treatment, it was found that more sustained growth inhibition was exerted by the combination. Pharmacodynamic studies on WU-386 tumors collected 4-hour post end-point treatment (4 weeks), from respective cohorts, demonstrated a decrease in p-ERK levels in tumors treated with either SHP2i alone or the SHP2i/CDK4/6i combination, and the combination led to a greater p-ERK inhibition than TNO155. This demonstrates that the combined inhibition of SHP2 and CDK4/6 is active and produces durable responses in patient derived models of NF1-associated MPNST, and represents a novel treatment strategy for patients with MPNST.

### Example 2

### In Vitro Analysis of TNO155 Single Agent and Combination of TNO155+Ribociclib in Native NF1-MPNST Cell Lines and Trametinib Resistant Lines

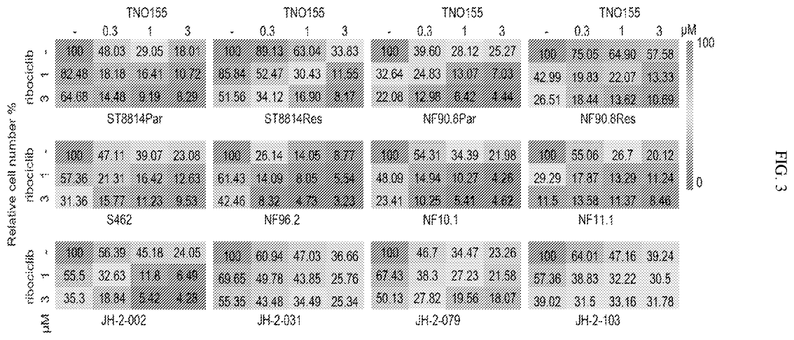

Ten native NF1-MPNST cell lines (ST8814Par, NF90.8Par, S462, NF96.2, NF10.1, NF11.1, JH-2-002, JH-2-031, JH-2-079 and JH-2-103) and two trametinib resistant lines (ST8814Res and NF90.8Res) were treated with DMSO, TNO155 (0.3, 1 and 3 μM), ribociclib (1 and 3 μM) or their combination for about 1 week. Cell numbers were counted using trypan blue exclusion assay (Sigma-Aldrich) and normalized to the DMSO control. FIG. 3 shows the results as a heat map. The ten native NF1-MPNST cell lines demonstrated partial sensitivity to TNO155 single agent, and deeper response was observed with the combination of TNO155+ribociclib, relative to TNO155 alone. TNO155 single agent proved to have limited activity, however, combination benefit was seen in the two MEKi-resistant cell line models.

### Example 3

### In Vitro Analysis of TNO155 Single Agent and Combination of TNO155+Ribociclib in Native NF1-MPNST Cell Lines and Trametinib Resistant Lines

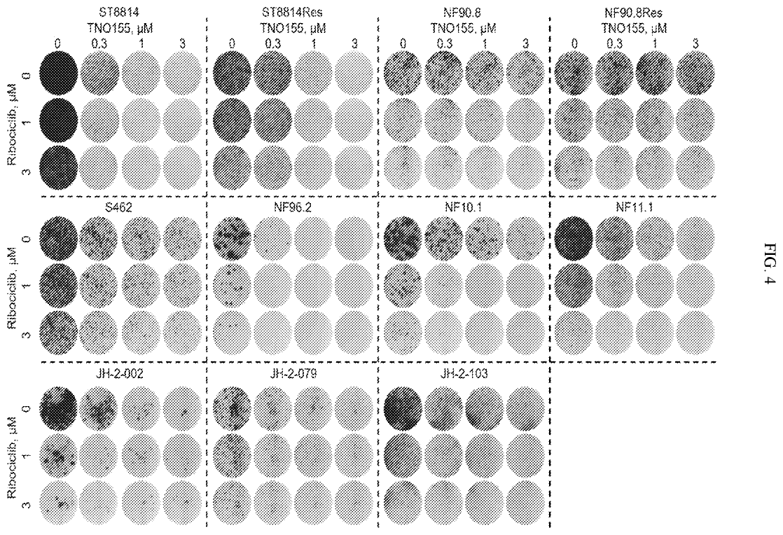

11 NF1-MPNST cell lines were treated with DMSO, TNO155 (0.3, 1 and 3 μM), ribociclib (1 and 3 μM) or their combination for about 2 weeks. Cells were washed with PBS, fixed with 10% neutral buffered formalin and then stained with 0.1% crystal violet. FIG. 4 shows that TNO155 and ribociclib have a combination benefit in several NF1-MPNST cell lines.

The combination of TNO155 and ribociclib has been investigated in in vitro cell line models of MPNST as well as in in vivo patient-derived xenograft (PDX) MPNST models. In cell line models, anti-tumor activity was observed with both single agents, which was enhanced in combination (see FIGS. 1-4). Mechanistically, the combination of TNO155 and ribociclib led to decreased ERK signaling and CDK4-cyclin DI activity compared to either drug alone. In in vivo patient-derived xenograft (PDX) models of NF1-associated MPNST, TNO155 displayed substantial anti-tumor activity as a single agent, which was enhanced in several models by the addition of ribociclib. The in vitro and in vivo observation indicates that combined use of TNO155 and ribociclic produces deeper and durable responses, and can overcome MEKi (trametinib) resistance. The combination of TNO155 and ribociclic may become a potential treatment approach for MPNST patients who have received MEK inhibitor treatment for their prior benign tumor neurofibromas and have developed acquired resistance, and for MPNST patients who exhibit intrinsic resistance to MEK inhibitor.

It is understood that the Examples and embodiments described herein are for illustrative purposes only and that various modifications or changes in light thereof will be suggested to persons skilled in the art and are to be included within the spirit and purview of this application and scope of the appended claims.

What is claimed is:

1. A method of treating a malignant peripheral nerve sheath tumor comprising administering to a patient in need thereof a therapeutically effective amount of a SHP2 inhibitor.

2. The method of claim 1 wherein the malignant peripheral nerve sheath tumor is metastatic, unresectable, sporadic, associated with neurofibromatosis type 1 or associated with radiotherapy.

3. The method of claim 1 or 2 in which the SHP2 inhibitor is (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof.

4. The method of claims 1-3 wherein (3S,4S)-8-(6-amino-5-((2-amino-3-chloropyridin-4-yl)thio)pyrazin-2-yl)-3-methyl-2-oxa-8-azaspiro[4.5]decan-4-amine is administered orally at a dose of about 1.5 mg per day, or 3 mg per day, or 6 mg per day, or 10 mg per day, or 20 mg per day, or 30 mg per day, or 40 mg per day, or 50 mg per day, or 60 mg per day, or 70 mg per day, or 80 mg per day, or 90 mg per day, or 100 mg per day.

5. The method of claims 1-4 wherein the dosing schedule is selected from continuous, 2 weeks on/1 week off or 3 weeks on/1 week off.

6. A method of treating a malignant peripheral nerve sheath tumor comprising administering to a patient in need thereof a pharmaceutical composition comprising: (a) a SHP2 inhibitor; and (b) a CDK4/6 inhibitor.

7. The method of claim 6 wherein the malignant peripheral nerve sheath tumor is sporadic, associated with neurofibromatosis type 1 or associated with radiotherapy.

8. The method of claim 6 or 7 in which the SHP2 inhibitor is (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof.

9. The method of claims 6-8 wherein (3S,4S)-8-(6-amino-5-((2-amino-3-chloropyridin-4-yl)thio)pyrazin-2-yl)-3-methyl-2-oxa-8-azaspiro[4.5]decan-4-amine is administered orally at a dose of about 1.5 mg per day, or 3 mg per day, or 6 mg per day, or 10 mg per day, or 20 mg per day, or 30 mg per day, or 40 mg per day, or 50 mg per day, or 60 mg per day, or 70 mg per day, or 80 mg per day, or 90 mg per day, or 100 mg per day.

10. The method of claims 6-9 wherein the dosing schedule is selected from continuous, 2 weeks on/1 week off or 3 weeks on/1 week off.

11. The method of claims 6-10 in which the CDK4/6 inhibitor is 7-cyclopentyl-N,N-dimethyl-2-((5-(piperazin-1-yl)pyridin-2-yl)amino)-7H-pyrrolo[2,3-d]pyrimidine-6-carboxamide, or pharmaceutically acceptable salt thereof.

12. The method of claims 6-11 wherein 7-cyclopentyl-N,N-dimethyl-2-((5-(piperazin-1-yl)pyridin-2-yl)amino)-7H-pyrrolo[2,3-d]pyrimidine-6-carboxamide is administered orally at a dose of about 100 mg per day, or 200 mg per day, or 300 mg per day, or 400 mg per day, or 500 mg per day, or 600 mg per day.

13. The method of claims 6-12 wherein 7-cyclopentyl-N,N-dimethyl-2-((5-(piperazin-1-yl)pyridin-2-yl)amino)-7H-pyrrolo[2,3-d]pyrimidine-6-carboxamide is administered orally at 600 mg for 21 days followed by 7 days off treatment.

14. A method of treating a malignant peripheral nerve sheath tumor comprising administering to a patient in need thereof a pharmaceutical composition comprising: (a) a MEK inhibitor; and (b) a CDK4/6 inhibitor.

15. The method of claim 14 wherein the malignant peripheral nerve sheath tumor is sporadic, associated with neurofibromatosis type 1 or associated with radiotherapy.

16. The method of claim 14 or 15 in which the MEK inhibitor is N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide, or pharmaceutically acceptable salt thereof.

17. The method of claims 14-16 wherein N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide is administered dimethyl sulfoxide per day is administered orally at a dose of about 0.5, 1, 1.5 and 2 mg daily.

18. The method of claims 14-17 in which the CDK4/6 inhibitor is 7-cyclopentyl-N,N-dimethyl-2-((5-(piperazin-1-yl)pyridin-2-yl)amino)-7H-pyrrolo[2,3-d]pyrimidine-6-carboxamide, or pharmaceutically acceptable salt thereof.

19. The method of claims 14-18 wherein 7-cyclopentyl-N,N-dimethyl-2-((5-(piperazin-1-yl)pyridin-2-yl)amino)-7H-pyrrolo[2,3-d]pyrimidine-6-carboxamide is administered orally at a dose of about 100 mg per day, or 200 mg per day, or 300 mg per day, or 400 mg per day, or 500 mg per day, or 600 mg per day.

20. The method of claims 14-19 wherein 7-cyclopentyl-N,N-dimethyl-2-((5-(piperazin-1-yl)pyridin-2-yl)amino)-7H-pyrrolo[2,3-d]pyrimidine-6-carboxamide is administered orally at 600 mg for 21 days followed by 7 days off treatment.
